(19) United States
(12) Patent Application Publication
(10) Pub. No.: US 2018/0325839 A1
(43) Pub. Date: Nov. 15, 2018

(54) TRANSDERMAL PATCHES

(71) Applicant: BUZZZ PHARMACEUTICALS LIMITED, Raheny Dublin (IE)

(72) Inventors: Ricky WING SING TSANG, Maidenhead, Berkshire (GB); Marc Barry BROWN, Watford, Hertfordshire (GB); Charles Rodney Greenaway EVANS, Worthing, Sussex (GB)

(21) Appl. No.: 15/777,481

(22) PCT Filed: Nov. 22, 2016
(86) PCT No.: PCT/EP2016/078479

§ 371 Date: May 18, 2018

(30) Foreign Application Priority Data
Nov. 23, 2015 (GB) 1520647.7

(51) Int. Cl. A61K 9/70

(52) U.S. Cl. CPC A61K 9/7069; A61K 31/445; A61K 9/7061

(57) ABSTRACT

The present invention relates to a transdermal patch comprising a pharmaceutical formulation, the formulation comprising ropivacaine or an opioid, a pharmaceutically-acceptable adhesive comprising up to 75% (w/w) of silicone or polyisobutylene and between 5 and 20 wt. % of an excipient mixture. The present invention also relates to methods of preparation of such a pharmaceutical formulation, as well as the use of such a transdermal patch in the treatment of pain (e.g. nociceptive and/or neuropathic pain).

### FIELD OF THE INVENTION

The present invention relates to transdermal patches. More specifically, the present invention relates to transdermal patches comprising an anaesthetic or analgesic for transdermal administration. The present invention also relates to processes for the preparation of these transdermal patches, as well as to the use of these transdermal patches for the topical treatment of pain (e.g. neuropathic and/or nociceptive pain).

### BACKGROUND OF THE INVENTION

Nociceptive pain is pain generated from nociceptors responding to stimuli by sending nerve signals to the spinal cord and brain. Such signals may be indicative of tissue irritation, impending injury, or actual injury, and are often characterized as aching and/or direct pains. Examples of conditions associated with nociceptive pain include bone fractures, burns, bumps, bruises, inflammation (from an infection or arthritic disorder), arthralgia, general myalgia and more specific myalgia caused by symptoms categorized generally as amplified musculoskeletal pain (AMP) syndrome.

Neuropathic pain is pain caused by damage or disease that affects the somatosensory system. Neuropathic pain is the result of an injury or malfunction in the peripheral or central nervous system. The pain is often triggered by an injury, but it is not necessary for such an injury to involve actual damage to the central nervous system. Nerves can be infiltrated or compressed by tumours, strangulated by scar tissue, or inflamed by infection. The pain is typically characterized by burning, lancinating, coldness or so-called pins-and-needles-type sensations. Persistent allodynia—pain resulting from a non-painful stimulus such as a light touch—is also a common characteristic of neuropathic pain. The pain itself may have continuous and/or episodic (paroxysmal) components, the having electric shock-like qualities. The pain may persist for months or years beyond the apparent healing of any damaged tissue. In these scenarios, such pain signals no longer represent an alarm about ongoing or impending injury, rather it is the alarm system itself that is malfunctioning. Common causes of painful peripheral neuropathies are herpes zoster, infection, HIV-related neuropathies, nutritional deficiencies, toxins, remote manifestations of malignancies, immune mediated disorders and physical trauma to a nerve trunk. Neuropathic pain is also common in cases of cancer, either as a direct result of a cancer on peripheral nerves (for example through compression by a tumour), or as a side effect of chemotherapy radiation, injury or surgery.

In certain conditions, the pain may be caused by a complex mixture of nociceptive and neuropathic factors. For example, myofascial pain is understood to result from nociceptive input from muscles. It is, however, plausible that such abnormal muscle activity is itself the result of neuropathic conditions.

In both neuropathic and nociceptive disease types, neurons become unusually sensitive and develop spontaneous activity, abnormal excitability, and a heightened sensitivity to chemical, thermal and mechanical stimuli. This phenomenon is known as “peripheral sensitization”. Localized delivery of anaesthetic can afford a method of desensitizing the aberrant stimuli.

Lidocaine (often referred to as lignocaine) is widely used as a local anaesthetic, and is commercially available in both an injectable form and as a transdermal patch. When compared with a systemic dose, transdermal delivery of local anaesthetics provides prolonged anaesthesia at the target site for pain suppression, and involves reduced plasma levels, hence a reduced potential toxicity.

However, in spite of the widespread use of lignocaine transdermal patches, there remains a need for improved transdermal anaesthetic formulations.

In addition, there remains a need for improved analgesic transdermal patch formulations to provide analgesia, in particular improved patches for the delivery of opioid analgesics.

There is also a need for transdermal formulations having good skin penetration properties. Moreover, there is a need for transdermal formulations of anaesthetic or analgesic agents that exhibit improved drug potency and having a longer duration of action for reducing the occurrence of breakthrough pain.

Furthermore, many transdermal formulations suffer from the viscoelastic migration of the adhesive from the edge of the patch during storage or application, a property known as cold flow. Cold flow can lead to a number of complications such as reduced adhesion during use, reduced drug potency, the adhesive sticking the inside of the packaging during storage and the formation of black rings on the skin during use. Thus, there also remains a need for transdermal formulations with reduced levels of cold flow.

Aspects of the invention were devised with the foregoing in mind.

### SUMMARY OF THE INVENTION

The present invention provides novel transdermal formulations suitable for topical application for the treatment of pain, for example nociceptive and/or neuropathic pain.

Thus, according to a first aspect of the invention, there is provided a transdermal patch comprising a pharmaceutical formulation, said formulation comprising:
(i) ropivacaine or an opioid,
(ii) a pharmaceutically-acceptable adhesive, comprising up to 75% (w/w) silicone or polyisobutylene, and
(iii) between 5 and 20% w/w of an excipient mixture comprising a hydrophilic material, a penetration enhancer and a carrier oil having a ropivacaine or an opioid solubility of greater than or equal to 1.5% (w/w).

In another aspect, the present invention provides a pharmaceutical formulation suitable for inclusion into a transdermal patch as herein defined, said formulation comprising ropivacaine or an opioid and a pharmaceutically-acceptable adhesive, and wherein said formulation has an in vitro human skin permeation rate greater than 1.8 μg cm⁻² h⁻¹.

In another aspect, the present invention provides a pharmaceutical formulation or transdermal patch as herein defined for use as a medicament.

In another aspect, the present invention provides a pharmaceutical formulation or transdermal patch as herein defined for use in the treatment of pain (e.g. neuropathic and/or nociceptive pain).

In another aspect, the present invention provides a method of treating pain (e.g. neuropathic and/or nociceptive pain), said method comprising topically administering to a human or animal subject in need of such treatment a therapeutically effective amount of a pharmaceutical formulation as defined herein, or applying a transdermal patch as herein defined.

In another aspect, the present invention provides a method of preparing a pharmaceutical formulation as defined herein, said method comprising mixing:
(i) ropivacaine or an opioid,
(ii) an adhesive as defined herein, and optionally
(iii) between 5 and 20 wt. % of an excipient mixture as defined herein, a hydrophilic material as defined herein, a penetration enhancer as defined herein and a carrier oil as herein defined.

### DETAILED DESCRIPTION OF THE INVENTION

### Ropivacaine

Ropivacaine, chemical name (2S)—N-(2,6-dimethylphenyl)-1-propyl-2-piperidinecarboxamide and having the structure shown below, is an aminoamide containing an asymmetric carbon atom, and is produced as the single S enantiomer for clinical use as local anaesthetic.

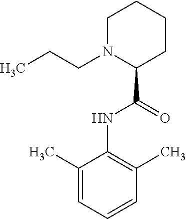 [SMILES: CCCN1CCCC[C@H]1C(=O)Nc1c(C)cccc1C]

Studies focussing on the use of local anaesthetic during cataract surgery have demonstrated that dose-for-dose, ropivacaine may be as much as four times as potent as lidocaine anaesthetics. In this study, the use of ropivacaine was preferred to lidocaine due to its longer half-life, which contributed to a reduction in levels of breakthrough pain.

In view of the above advantages, ropivacaine presents a suitable candidate for inclusion into a transdermal patch for the treatment of pain, such as nociceptive and neuropathic pain. In theory, such advantages would allow for a transdermal patch having improved drug potency and enhanced drug persistence characteristics.

However, in spite of the advantages discussed above, ropivacaine saturated H₂O has been demonstrated to be exhibit significantly poorer skin permeation characteristics than lidocaine saturated H₂O, thereby presenting a considerable barrier to transdermal patch development.

When used in conjunction with the present invention, ropivacaine may be present in its free base form, or as a salt. Suitably, when used as part of the pharmaceutical formulation described herein, ropivacaine is present in its free base form, since it is commonly understood that the skin is typically more permeable to uncharged lipophilic permeants, as opposed to charged species. The free base form would also be expected to be more soluble in typical pharmaceutical adhesives than would a salt form (e.g. ropivacaine HCl).

The amount of ropivacaine present in the pharmaceutical formulation of the present invention will depend on how soluble it is in the pharmaceutically-acceptable adhesive and excipients present. Typically, the ropivacaine will present at an amount of 3-20% w/w relative to total weight of the pharmaceutical formulation (i.e. the drug containing layer incorporated into the transdermal patch). In one embodiment, the amount of ropivacaine is between 3 and 15% w/w relative to total weight of the pharmaceutical formulation. Suitably, the amount of ropivacaine is between 3 and 12% w/w relative to total weight of the pharmaceutical formulation. More suitably, the amount of ropivacaine is between 6 and 10% w/w relative to total weight of the pharmaceutical formulation.

In a particular embodiment, the amount of ropivacaine present in the pharmaceutical formulation is 7 to 9% w/w or about 8% w/w relative to total weight of the pharmaceutical formulation.

### Opioid Analgesics

The transdermal patches of the present invention may comprise an opioid analgesic. Any suitable opioid analgesic may be used.

In an embodiment, the opioid analgesic is selected from morphine, codeine, thebaine, diacetylmorphine (morphine diacetate; heroin), nicomorphine (morphine dinicotinate), dipropanoylmorphine (morphine dipropionate), desomorphine, acetylpropionylmorphine, dibenzoylmorphine, diacetyldihydromorphine, hydromorphone, hydrocodone, oxycodone, oxymorphone, ethylmorphine and buprenorphine, fentanyl, pethidine, levorphanol, methadone, tramadol and dextropropoxyphene.

In a further embodiment, the opioid analgesic is oxycodone.

The amount of opioid present in the pharmaceutical formulation of the present invention will depend on how soluble it is in the pharmaceutically-acceptable adhesive and excipients present. Typically, the opioid will be present at an amount of 3-20% w/w relative to total weight of the pharmaceutical formulation.

In one embodiment, the amount of opioid is between 3 and 15% w/w relative to total weight of the pharmaceutical formulation.

Suitably, the amount of opioid is between 3 and 12% w/w relative to total weight of the pharmaceutical formulation. More suitably, the amount of opioid is between 6 and 10% w/w relative to total weight of the pharmaceutical formulation.

### Transdermal Patch

Despite a saturated solution of ropivacaine demonstrating poor in vivo skin penetration compared to a saturated solution of lidocaine, transdermal patches comprising ropivacaine have been developed and are described in International Patent Publication No WO2015052183.

One problem observed with some of the transdermal formulations described in WO2015052183 is that the phenomenon of cold flow has been observed to occur.

The formulations of the present invention are stable and demonstrate excellent levels of deliverability and good adhesive properties. They also demonstrate little or no cold flow.

The transdermal patches of the present invention are prepared by casting a wet formulation layer as described herein at a known thickness onto a suitable release liner. In its simplest form, the pharmaceutical formulation may comprise ropivacaine or an opioid and a pharmaceutically acceptable adhesive. The pharmaceutical formulation additionally comprises an excipient mixture, comprising one or more carrier oils, penetration enhancers and hydrophilic materials. Typically, the pharmaceutical formulation are cast at a wet thickness of between about 240 μm to about 550 μm, to provide a dry thickness of between about 45 μm and about 95 μm, suitably between about 80 μm and about 85 μm. After casting, the layer is dried, and then laminated with a backing membrane. A suitable container or closure system may be used protect the transdermal patch during transportation and storage.

Suitable backing membranes may be occlusive or non-occlusive. Where a non-occlusive backing membrane is used, it is desirable to use a fully occlusive container or closure system to prevent degradation of the cast pharmaceutical formulation layer prior to use. The backing membrane may be of any thickness, but is suitably between about 10 to 260 μm thick. Suitable materials include, but are not limited to, synthetic polymers including, for example, polyesters, polycarbonates, polyimides, polyethylene, poly(ethylene teraphthalate), polypropylene, polyurethanes and polyvinylchlorides. The backing membrane may also be a laminate comprising additional layers that may include vapour deposited metal, such as aluminium, additional synthetic polymers, and other materials, to enable a heat seal, such as EVA copolymer. Suitably, the backing membrane comprises occlusive Scotchpak 9730® obtainable from 3M.

The release liner is typically disposed on an opposite surface of the pharmaceutical formulation layer to the backing membrane and provides a removable protective or impermeable layer, usually, but not necessarily, rendered non-stick so as to not adhere to the pharmaceutical formulation layer. The release liner serves to protect the pharmaceutical formulation layer during storage and transit, and is intended to be removed prior to use. The release liner may be formed from the same materials used for the backing membrane, but in particular it may be formed from metal foils, Mylar®, polyethylene terephthalate, siliconized polyester, fumed silica in silicone rubber, polytretrafluoroethylene, cellophane, siliconized paper, aluminized paper, polyvinyl chloride film, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene, and styrene-isoprene copolymers, polyethylene, and polypropylene.

Suitably, the release liner is an occlusive or semi-occlusive backing film being compatible with the pharmaceutically-acceptable adhesive present in the pharmaceutical formulation layer.

Suitably, the release liner may be selected from Scotchpak 9741®, Scotchpak 1022®, Scotchpak 9742®, Scotchpak 9744®, Scotchpak 9748® and Scotchpak 9755®, all of which are obtainable from 3M and comprise fluoropolymers coated onto polypropylene or polyester film. Other suitable release liners made by other manufacturers may also be used. The release liner may be of any thickness known in the art. Suitably the release liner has a thickness of about 0.01 mm to about 2 mm.

In one embodiment, the release liner is Scotchpak 9741®. In another embodiment, the release liner is Scotchpak 1022®.

The container or closure system may be made from a range of materials suitable for protecting the packaged transdermal patch from moisture and light.

### Permeation Rate of Ropivacaine or Opioid

As previously stated, the present invention provides a transdermal patch comprising a pharmaceutical formulation, said formulation comprising:
(i) ropivacaine or an opioid;
(ii) a pharmaceutically-acceptable adhesive that comprises up to 75% (w/w) silicone or polyisobutylene; and
(iii) between 5 and 20 wt. % of an excipient mixture comprising a hydrophilic material, a penetration enhancer and a carrier oil,
   and wherein said formulation has an in vitro human skin permeation rate of ropivacaine or opioid that is greater than 1.8 μg cm⁻² h⁻¹.

Permeation/release measurements of ropivacaine or opioid through a 9% EVA membrane were used as a tool to select candidate patches. Permeation/release data was only recorded for those patches that remained free of drug precipitation (i.e. those that were below saturation concentration).

The present invention also provides a pharmaceutical formulation, said formulation comprising ropivacaine or opioid, a pharmaceutically-acceptable adhesive comprising up to 75% (w/w) silicone, and between 5 and 20 wt. % of an excipient mixture comprising a hydrophilic material, a penetration enhancer and a carrier oil, and wherein said formulation has an in vitro human skin permeation rate of ropivacaine or opioid that is greater than 1.8 μg cm⁻² h⁻¹.

By in vitro human skin permeation rate we mean the rate of delivery of ropivacaine or opioid through human epidermal membranes at time periods up to 12 hours.

Suitably, the in vitro human skin permeation rate of ropivacaine or opioid is the apparent steady state flux (calculated from the approximately linear portion of the cumulative permeation profile), typically observed between 3 and 12 hours, or between 4 and 12 hours, when assessed under the conditions detailed in the following sections.

In an embodiment, the in vitro human skin permeation rate of ropivacaine or opioid is between 1.8 μg cm⁻² h⁻¹ and 10 μg cm⁻² h⁻¹.

In a further embodiment, the in vitro human skin permeation rate of ropivacaine or opioid is between 2 μg cm⁻² h⁻¹ and 6 μg cm⁻² h⁻¹.

In a further embodiment, the in vitro human skin permeation rate of ropivacaine or opioid is between 3 μg cm⁻² h⁻¹ and 5 μg cm⁻² h⁻¹.

### Pharmaceutically-Acceptable Adhesive

The pharmaceutically-acceptable adhesive is selected both in terms of its ability to solubilise ropivacaine or an opioid, and its adhesive tack and peel properties. The inventors surprisingly found that adhesives comprising up to 75% (w/w with respect to the other components of the adhesive) silicone gave good drug deliverability and low levels of cold flow.

In one embodiment, the adhesive has a ropivacaine or opioid solubility in excess of 2.5% w/w at room temperature.

In an embodiment, the adhesive is an acrylate material, optionally blended with one or more rubbers, which comprises up to 75% (w/w) silicone or polyisobutylene. Suitably, the adhesive a blend of acrylate materials comprising up to 75% (w/w) silicone or polyisobutylene. More suitably, the adhesive is a blend of acrylate materials and/or rubbers comprising up to 75% (w/w) silicone. Even more suitably, the adhesive a blend of acrylate materials comprising up to 75% (w/w) silicone.

In another embodiment, the adhesive is a blend of acrylate materials and silicone, wherein the ratio of acrylate material:silicone is between 95:5 to 25:75. Suitably, the ratio of acrylate material:silicone is between 90:10 to 30:70. More suitably, the ratio of acrylate material:silicone is between 90:10 to 40:60. Even more suitably, the ratio of acrylate material:silicone is between 75:25 to 40:60. Most suitably, the ratio of acrylate material:silicone is about 50:50.

It will be understood that the acrylate material may include acrylate copolymers and acrylate-vinyl acetate, such as Duro-Tak 87-2677®, Duro-Tak 87-900A®, Duro-Tak 87-2074®, Duro-Tak 87-2054®, Duro-Tak 87-2052®, Duro-Tak 87-2196®, obtainable from Henkel.

In another embodiment, the acrylate is selected from Duro-Tak 87-900A®, Duro-Tak 87-2677® and Duro-Tak 87-2074®, Duro-Tak 387-2054® or Duro-Tak 87-2852®. Suitably, the acrylate/polyacrylate is selected from Duro-Tak 387-2054® or Duro-Tak 87-2852®.

Any suitable silicone may be used in the adhesive of the present invention. In an embodiment, the silicone is polydimethylsiloxane. Suitably, silicone is a polydimethylsiloxane selected from Bio PSA 7-4401®, Bio-PSA 7-4402®, Bio PSA 7-4501®, Bio PSA 7-4502®, Bio PSA 7-4601®, Bio PSA 7-4602®, Bio PSA 7-4101®, Bio PSA 7-4102®, Bio PSA 7-4201®, Bio PSA 7-4202®, Bio PSA 7-430®, Bio PSA 7-4302® or Bio PSA 7-4560®. More suitably, silicone is a polydimethylsiloxane selected from Bio PSA 7-4101®, Bio PSA 7-4102®, Bio PSA 7-4201®, Bio PSA 7-4202®, Bio PSA 7-430® or Bio PSA 7-4302®. Most suitably, the silicone is Bio PSA 7-4302®.

In another embodiment, the adhesive is a 50:50 blend of Duro-Tak 387-2054® or Duro-Tak 87-2852® and Bio PSA 7-4302®.

In yet another embodiment, a suitable volatile solvent is added to the adhesive to reduce viscosity and aid solvation. Suitable solvents may include, but are not limited to, isopropyl alcohol, methanol, ethanol and ethyl acetate.

Typically, the amount of adhesive present in the pharmaceutical formulation (i.e. the drug containing layer of the transdermal patch) is between 60 and 92% w/w. Suitably, the amount of adhesive is between 70 and 92% w/w. More suitably, the amount of adhesive is between 73 and 87% w/w. Most suitably, the amount of adhesive is between 80 and 85% w/w (for example, about 82%).

### Excipient Mixture

In addition to the drug (ropivacaine or an opioid), the pharmaceutical formulation comprises 5 to 20% w/w of an excipient mixture comprising a hydrophilic material, a penetration enhancer and a carrier oil.

Without wishing to be bound by any particular theory, it is believed that the excipient mixture of the present invention improves the transdermal delivery of the ropivacaine or opioid by the temporary alteration of the skin barrier function, or by improvements in drug/skin partitioning resulting from increased solubility of the drug in the stratum corneum. The selection of excipient mixtures is designed to maintain reasonable solubility of the ropivacaine or opioid in the pharmaceutically-acceptable adhesive. It is not necessary for the excipient mixture to increase drug solubility in the pharmaceutically-acceptable adhesive. In certain embodiments the solubility of the ropivacaine or opioid in the selected excipient mixture is greater than the solubility of the ropivacaine or opioid in each individual excipient. In such embodiments, the observed solubility is significantly greater than the predicted solubility based upon proportional contributions from the solubilities in individual excipients, suggesting a significant cooperative effect on drug solubility.

The inclusion of one or both of a penetration enhancer and/or a hydrophilic material in the binary or ternary mixtures may contribute to improving transdermal ropivacaine or opioid delivery by increasing skin permeation according to the mechanisms discussed in the preceding paragraphs.

The inventors advantageously and surprisingly found that reducing the amount of excipient mixture present in the pharmaceutical formulation resulted in a decreased cold flow being observed, whilst maintaining good levels of drug deliverability.

Thus, the pharmaceutical formulation present in the transdermal patches of the present invention comprise between 5 and 20% w/w (relative to the total weight of the pharmaceutical formulation) of an excipient mixture. Suitably, the excipient mixture is present in an amount of between 5 and 18% w/w. More suitably, the excipient mixture is present in an amount of between 8 and 18% w/w. Even more suitably, the excipient mixture is present in an amount of between 8 and 16% w/w. Most suitably, the excipient mixture is present in an amount of about 10 to 15% w/w (for example about 10 wt. %).

The relative amounts of hydrophilic material, penetration enhancer and carrier oil present within an excipient mixture may vary. In an embodiment, the excipient mixture comprises the following composition:

| | |
|---|---|
| hydrophilic | 10-40% w/w (relative to the total weight of the excipient |
| material | mixture) |
| penetration | 5-30% w/w (relative to the total weight of the excipient |
| enhancer | mixture) |
| carrier oil | 30-80% w/w (relative to the total weight of the excipient |
| | mixture) |

In another embodiment, the excipient mixture comprises the following composition:

| | |
|---|---|
| hydrophilic | 20-40% w/w (relative to the total weight of the excipient |
| material | mixture) |
| penetration | 5-15% w/w (relative to the total weight of the excipient |
| enhancer | mixture) |
| carrier oil | 50-80% w/w (relative to the total weight of the excipient |
| | mixture) |

In another embodiment, the excipient mixture comprises the following composition:

| | |
|---|---|
| hydrophilic | 25-40% w/w (relative to the total weight of the excipient |
| material | mixture) |
| penetration | 5-15% w/w (relative to the total weight of the excipient |
| enhancer | mixture) |
| carrier oil | 50-70% w/w (relative to the total weight of the excipient |
| | mixture) |

In particular embodiment, the excipient mixture comprises the following composition:

| | |
|---|---|
| hydrophilic | about 30% w/w (relative to the total weight of the excipient |
| material | mixture) |
| penetration | about 10% w/w (relative to the total weight of the excipient |
| enhancer | mixture) |
| carrier oil | about 60% w/w (relative to the total weight of the excipient |
| | mixture) |

In one embodiment, the excipient mixture only comprises a hydrophilic material, a penetration enhancer and a carrier oil.

In another embodiment, other components may be present. For example, in order to further optimise the properties of the pharmaceutical formulations of the present invention, such as skin penetration, volatility and adhesion, the excipient mixture may further comprise one or more additives selected from non-ionic surfactants, hydrophilic surfactants, terpenes and dual membrane disruptors, including those obtainable under the trade names Transcutol®, Brij 98®, Tween 80®, Cremphor EL® and menthol.

### Carrier Oil

The carrier oil is selected both for its compatibility with the pharmaceutically-acceptable adhesive and for its ability to solubilise ropivacaine or the opioid. Carrier oils used in conjunction with the present invention include, but are not limited to, sorbitan monooleate, sorbitan trioleate, triglycerides of caprylic/capric acid, propylene glycol dicaprylate/dicaprate, ethoxy diglycol, propylene glycol monocaprylate, glycerol monooleate, lanolin, acetylated lanolin, polyethylene glycol lanolin, glycerol monocaprylate/caprate, propylene glycol laurate, and/or mono- or diglycerides of capric acid.

Suitably, the carrier oil has a water solubility of less than 0.1% (w/w) and a ropivacaine or opioid solubility in excess of 3% (w/w).

Suitably, the carrier oil may be sorbitan trioleate, propylene glycol monocaprylate, glycerol monocaprylate/caprate, propylene glycol laurate, and/or mono- or diglycerides of capric acid. Suitably, the carrier oil is present in the pharmaceutical formulation at an amount of 1% (w/w) to 20% (w/w). In an embodiment, the carrier oil is in an amount of 1 and 15% w/w. In another embodiment, the carrier oil is in an amount of 2 and 12% w/w. In another embodiment, the carrier oil is in an amount of 4 and 10% w/w. In another embodiment, the carrier oil is in an amount of 5 and 10% w/w. In another embodiment, the carrier oil is in an amount of about 8% w/w.

Suitably, the carrier oil has a ropivacaine or opioid solubility in excess of 4% (w/w).

Suitably, the carrier oil may be propylene glycol monocaprylate, propylene glycol laurate and/or mono- or diglycerides of capric acid. Even more suitably, the carrier oil is propylene glycol monocaprylate, obtainable under the trade name Capryol 90®.

### Penetration Enhancer

The penetration enhancers used in the pharmaceutical formulations and transdermal patches of the present invention serve to promote the percutaneous absorption of the ropivacaine or opioid by temporarily diminishing the impermeability of the skin. Importantly, when included in the pharmaceutical formulations of the present invention, the penetration enhancer must not compromise the release characteristics of the adhesive.

Suitably, the penetration enhancer and the quantities in which it is added should be non-toxic, non-irritating, non-allergenic, odourless, tasteless, colourless, soluble, and compatible with ropivacaine or the opioid and the other excipients herein described. Importantly, the enhancer should not lead to the loss of bodily fluids, electrolytes and other endogenous materials, and skin should immediately regain its barrier properties on its removal. Examples of penetration enhancers suitable for inclusion into the pharmaceutical formulation of the present invention include, but are not limited to, sugar fatty acid esters and ethers, C₈-C₁₈ fatty alcohols and their ester derivatives, azone, oleic ethers, terpenes and ethoxy ethanols.

The penetration enhancer may be present in the pharmaceutical formulation in an amount of about 0.25% w/w to 15% w/w (relative to the weight of the total pharmaceutical formulation, i.e. the drug-containing layer of the transdermal patch).

Suitably, the penetration enhancer is present in the pharmaceutical formulation in an amount of about 0.25% w/w to 10% w/w, more suitably,

In an embodiment, the penetration enhancer is present in the pharmaceutical formulation in an amount of 0.25% w/w to 8% w/w. In another embodiment, the penetration enhancer is present in the pharmaceutical formulation in an amount of 0.25% w/w to 6% w/w. In another embodiment, the penetration enhancer is present in the pharmaceutical formulation in an amount of 0.5% w/w to 4% w/w. In another embodiment, the penetration enhancer is present in the pharmaceutical formulation in an amount of 0.5% w/w to 2% w/w. In another embodiment, the penetration enhancer is present in the pharmaceutical formulation in an amount of 0.5% w/w to 1.5% w/w.

In a particular embodiment, the penetration enhancer is present in the pharmaceutical formulation in an amount of about 1% w/w.

Any suitable penetration enhancer may be used. For example, the penetration enhancer may be a polyoxyethylene oleyl ether, obtainable under the trade name Brij 93®, or 2-(2-ethoxyethoxy)ethanol, obtainable under the trade name Transcutol®, or menthol.

In one embodiment, the penetration enhancer is polyoxyethylene oleyl ether or a compound of the formula I, shown below:

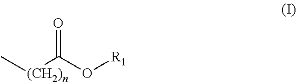

wherein;
R₁ is a (1-6C)alkyl; and
n is an integer of between 6 and 20.

In another embodiment, the penetration enhancer is a compound of the formula I, shown below:

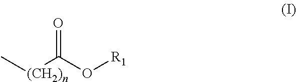

wherein;
R₁ is a (1-6C)alkyl; and
n is an integer of between 6 and 20.

Suitably, where the penetration enhancer is a compound of the formula I, R₁ and n may have any of the meanings defined hereinbefore or as defined in any of paragraphs (1) to (6) hereinafter:—

(1) R₁ is a (1-4C)alkyl;

(2) R₁ is a (2-4C)alkyl;

(3) R₁ is isopropyl;

(4) n is an integer of between 10 and 20;

(5) n is an integer of between 12 and 18; or

(6) n is an integer of between 14 and 16.

In another embodiment, the penetration enhancer is polyoxyethylene oleyl ether or isopropyl palmitate. Suitably, the penetration enhancer is isopropyl palmitate.

In another embodiment, the penetration enhancer is included in the pharmaceutical formulation as part of an excipient mixture including a carrier oil and a hydrophilic material.

In another embodiment, the excipient mixture may comprise two or more penetration enhancers as defined herein.

### Hydrophilic Material

The hydrophilic material used in the present invention is believed to aid the skin absorption of the ropivacaine or opioid. The hydrophilic material may be present as a polar enhancer, and is liquid at skin temperature. Suitably, the hydrophilic material and the quantities in which it is added should be non-toxic, non-irritating, non-allergenic, odourless, tasteless, colourless, soluble, and compatible with the ropivacaine or opioid and the other excipients herein described.

In one embodiment, the hydrophilic material will have a hydrophilic-lipophilic balance (HLB) of greater than 7. Examples of hydrophilic materials suitable for inclusion into the pharmaceutical formulation of the present invention include, but are not limited to, propylene glycol, glycerol, polyethylene glycol, short chain water soluble esters of citric acid, acetic acid, hexylene glycol and alcohols, including diols and polyols.

In an embodiment, the hydrophilic material is propylene glycol.

Suitably, the hydrophilic material is present in the pharmaceutical formulation in an amount of about 1% w/w to about 15% w/w (relative to the weight of the total pharmaceutical formulation, i.e. the drug-containing layer of the transdermal patch). More suitably, the hydrophilic material is present in the pharmaceutical formulation in an amount of about 1% w/w to about 10% w/w. In an embodiment, the hydrophilic material is present in the pharmaceutical formulation in an amount of about 1% w/w to about 6% w/w. In another embodiment, the hydrophilic material is present in the pharmaceutical formulation in an amount of about 1% w/w to about 4% w/w. In another embodiment, the hydrophilic material is present in the pharmaceutical formulation in an amount of about 2% w/w to about 4% w/w. In a particular embodiment, the hydrophilic material is present in the pharmaceutical formulation in an amount of about 3% w/w.

### PARTICULAR EMBODIMENTS

In an embodiment, the pharmaceutical formulations present in the transdermal patches of the present invention have the following composition:

3-20% w/w of ropivacaine or opioid

60-92% w/w of adhesive

5-20% w/w of excipient mixture.

In an embodiment, the pharmaceutical formulations present in the transdermal patches of the present invention have the following composition:

3-15% w/w of ropivacaine or opioid

70-92% w/w of adhesive

5-15% w/w of excipient mixture.

In an embodiment, the pharmaceutical formulations present in the transdermal patches of the present invention have the following composition:

3-12% w/w of ropivacaine or opioid

73-84% w/w of adhesive

10-15% w/w of excipient mixture.

In a further embodiment, the pharmaceutical formulations present in the transdermal patches of the present invention have the following composition:
6-12% w/w of ropivacaine or opioid;
30-50% w/w acrylate adhesive (e.g. Durotak 387-2054 or Durotak 87-2852)
30-50% w/w of silicone adhesive (e.g. Bio-PSA 7-4302);
10-15% w/w of excipient mixture (e.g. propylene glycol/Capryol 90/Brij93 [30/60/10] or propylene glycol/Capryol 90/isopropyl palmitate [30/60/10]).

In a further embodiment, the pharmaceutical formulations present in the transdermal patches of the present invention have the following composition:
8-10% w/w of ropivacaine or opioid;
35-45% w/w acrylate adhesive (e.g. Durotak 387-2054 or Durotak 87-2852)
35-45% w/w of silicone adhesive (e.g. Bio-PSA 7-4302);
8-10% w/w of excipient mixture (e.g. propylene glycol/Capryol 90/Brij93 [30/60/10] or propylene glycol/Capryol 90/isopropyl palmitate [30/60/10]).

### Preparation of Pharmaceutical Formulations

The pharmaceutical formulations of the present invention can be prepared using conventional techniques known in the art.

The pharmaceutical formulations are suitably prepared by mixing all of the components together.

The individual components may be mixed by simply adding all of the components at the same time into a mixing vessel and then mixing them all together (a “one-pot” mixture). Alternatively, the components may be added sequentially in two or more steps or stages. Suitably, the excipient mixture is pre-mixed prior to mixing with the other components of the formulation.

Other experimental conditions required to prepare the formulations of the present invention, such as mixing times, mixing equipment, temperature control etc. can be readily determined by a person of ordinary skill in the art.

Further experimental details will also be evident from the accompanying Examples.

Once prepared, the pharmaceutical formulations of the present invention are incorporated into a transdermal patch for topical application.

### Therapeutic Uses

The pharmaceutical formulations of the present invention are particularly suited to the treatment of pain. Once administered, the transdermal patch comprising the pharmaceutical formulation provides a localised delivery of the ropivacaine or opioid, thus providing pain relief at a desired location. During localised delivery, quantities of the ropivacaine or opioid may be absorbed into the patient's blood stream, thereby providing an additional, systemic delivery of the anaesthetic.

Types of pain that can be treated with the transdermal patch of the present invention include nociceptive and neuropathic pain.

Nociceptive pain may be pain associated with tissue irritation, impending injury, or actual injury, and is often characterized as aching and/or direct pains. Examples of conditions associated with nociceptive pain include bone fractures, burns, bumps, bruises, inflammation (from an infection or arthritic disorder), arthralgia, general myalgia and more specific myalgia caused by symptoms categorized generally as amplified musculoskeletal pain (AMP) syndrome.

Neuropathic pain is pain caused by damage or disease that affects the somatosensory system. The pain is typically characterized by burning, lancinating, coldness or so-called pins-and-needles-type sensations. Persistent allodynia—pain resulting from a non-painful stimulus such as a light touch—is also a common characteristic of neuropathic pain. The pain itself may have continuous and/or episodic (paroxysmal) components, the having electric shock-like qualities. Common causes of painful peripheral neuropathies that can be treated with the transdermal patches of the present invention include herpes zoster, infection, HIV-related neuropathies, nutritional deficiencies, toxins, remote manifestations of malignancies, immune mediated disorders and physical trauma to a nerve trunk. Neuropathic pain is also common in cases of cancer, either as a direct result of a cancer on peripheral nerves (for example through compression by a tumour), or as a side effect of chemotherapy radiation, injury or surgery.

The transdermal patches of the present invention may also prove effective in cases where the pain is be caused by a complex mixture of nociceptive and neuropathic factors, for example, myofascial pain.

The pharmaceutical compositions of the present invention may be used on their own as the sole therapy. Alternatively, the compositions may be administered as part of a combination therapy with one or more other pain treatments or anaesthetics. Such conjoint treatment may be achieved by way of the simultaneous, sequential or separate administration of the individual components of the treatment.

### DETAILED DESCRIPTION OF THE DRAWINGS

The present invention is further defined with reference to the accompanying figures, in which data are presented as mean±standard error (SE), and where:

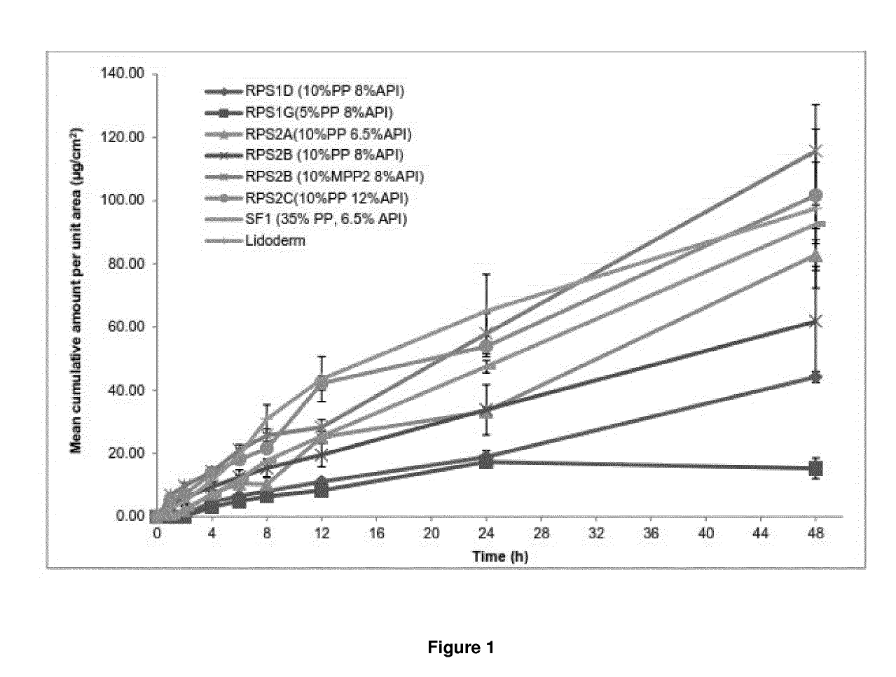

FIG. 1 shows the mean cumulative amount of ropivacaine released per unit area (n/cm²) from 6 formulations through 9% EVA membrane over t=48 h. Each time point represents the mean±SEM (n=5).

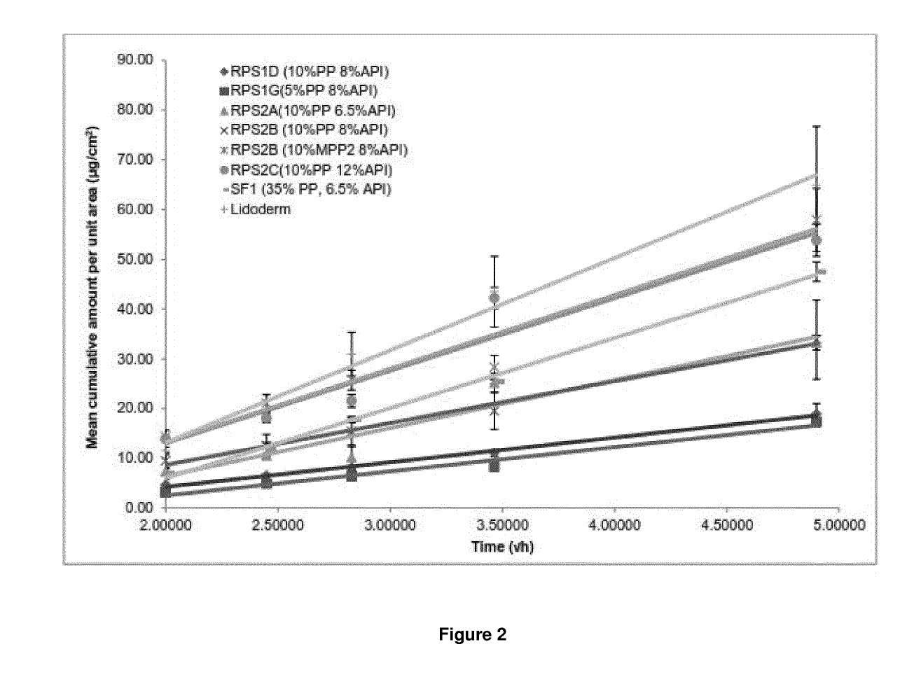

FIG. 2 shows the mean cumulative amount of ropivacaine released per unit area (n/cm²) from 6 formulations through 9% EVA membrane between 4-24 h. Each time point represents the mean±SEM (n=5).

### EXAMPLES

All formulations were prepared with 4 g of adhesive. The loadings of other constituents (prepared as w/w), such as excipient mixture, were adjusted for percentage solids of adhesive, such that the patch loadings were relative to the dry adhesive weight.

Ropivacaine was weighed into a single vessel. The one or more excipients were added followed by the adhesive, and the vessel was capped. Isopropyl alcohol was added before the addition of the adhesive for preparations using Duro-Tak® 87-2677. The vessel contents were then mixed using a roller mixer until a homogeneous mixture was obtained, and the ropivacaine was fully dissolved. Casting thicknesses were adjusted to account for the inclusion of the excipient mixture where appropriate. The mixtures were cast onto ScotchPak 9744® release liner, dried and then laminated with ScotchPak 9730® backing membrane to give patches with a thickness of 80 μm

To address the cold flow properties displayed by prior art formulations, the inventors surprisingly found that changing the adhesive composition (such that it comprises silicone) and the amount of the excipient mixture resulted in enhanced drug release and lower levels of cold flow.

### Example 1—Adhesive Blends

### Cold Flow Assessment

A selection of mixed adhesives based upon mixtures of acrylate and silicone adhesives were investigated for use in transdermal formulations of the present invention.

Preliminary patches containing either Duro-tak 387-2054 or Duro-tak 87-2852 were developed with varying levels of silicone adhesive (Bio PSA 7-4302) and excipient mixture (PG:Cap90:Brij93, 30:60:10). Cold flow assessment was performed on the preliminary patches, wherein the patches were applied to the skin for 12 hours, before the relative amounts of adhesive remaining on the skin after the allotted time were recorded after the patch was removed. The amount of remaining adhesive left on the skin was then used as a measure of the degree of cold flow of the patches. Table 1 shows the theoretical dried compositions (% w/w) of the patches and the cold flow results. The main findings from this assessment are as follows:
The general trend for both all patches assessed was that the lower the excipient mixture amount, the lower the level of cold flow that was observed.
In preliminary patches containing either Duro-tak 387-2054 or Duro-tak 87-2852, the initial observations showed that there was a low-medium level of cold flow in the majority of patches.
The cold flow assessments identified that higher levels of the excipient mixture incorporated into patches resulted in increased cold flow, with the optimal amount of excipient mixture found to be between 10-15%. In addition, it was observed that higher levels of excipient mixture (up to 15%) could be incorporated into patches with lower levels of silicone (90:10 acrylate:silicone) before cold flow was evident.

TABLE 1
Theoretical composition of placebo patches and macroscopic observations.

| | | Theoretical dried composition (% w/w) | | | | | | | | | | |
|---|---|---|---|---|---|---|---|---|---|---|---|---|
| | | RPS1C | RPS1D | RPS1D | RPS1G | RPS2B | RPS2C | RPS2C | RPS2C | RPS2D | RPS2D | RPS2D |
| Formulation | | 10% PP | 10% PP | 5% PP | 5% PP | 10% PP | 20% PP | 15% PP | 10% PP | 20% PP | 15% PP | 10% PP |
| Acrylate | Durotak | 67.50 | 81.00 | 85.50 | 90.25 | — | — | — | — | — | — | — |
| adhesive | 387-2054 | | | | | | | | | | | |
| | Durotak | — | — | — | — | 45.00 | 60.00 | 63.75 | 67.50 | 72.00 | 76.50 | 81.00 |
| | 87-2852 | | | | | | | | | | | |
| Silicone | Bio-PSA | 22.50 | 9.00 | 9.50 | 4.75 | 45.00 | 20.00 | 21.25 | 22.50 | 8.00 | 8.50 | 9.00 |
| | 7-4302 | | | | | | | | | | | |

| | | | | | | | | | | | |
|---|---|---|---|---|---|---|---|---|---|---|---|
| excipient | 10.00 | 10.00 | 5.00 | 5.00 | 10.00 | 20.00 | 15.00 | 10.00 | 20.00 | 15.00 | 10.00 |
| mixture (PP) | | | | | | | | | | | |
| Macroscopic | Turbid | Turbid | Turbid | Turbid | Turbid | Turbid | Turbid | Turbid | Turbid | Turbid | Turbid |
| appearance | premix | premix | premix | premix | premix | premix | premix | premix | premix | premix | premix |
| | and | and final | and final | and final | and | and final | and final | and final | and final | and final | and final |
| | final | patch | patch | patch | final | patch | patch | patch | patch | patch | patch |
| | patch | | | | patch | | | | | | |
| Cold flow | Med | Low-med | Low-med | Low-med | Low | Low-med | Low-med | Low | Low-med | Low | Low |
| assessment | | | | | | | | | | | |

### Short Term Physical Stability of Ropivacaine in Adhesive Blends

The stability of ropivacaine was determined by incorporating ropivacaine at 10% w/w in the adhesive blends described herein and observing for drug crystal formation under polarised light following preparation of the patch. Patches where no drug crystals were observed were then assessed for short term stability with observations of crystal formation conducted at t=1 and 2 week time points

Transdermal patches at varying levels of ropivacaine and adhesive blends were prepared as described above and those that were free of drug crystals were placed onto short-term stability tests at 2-8° C. for t=1 and 2 weeks, wherein microscopic observations were made at t=1 week and t=2 weeks to assess for ropivacaine crystal formation.

The short-term stability tests were carried out as detailed below:
(i) Ropivacaine was mixed into the appropriate adhesive system and stirred until the premix was observed to be clear and homogenous.
(ii) The selected release liner (ScotchPak 9744) was fitted into the pin frame of the MATHIS laboratory coating device type LTSV and the clocks were set on the coating device to achieve a nominal dried adhesive thickness of 80 μm, according to SOP 3155.
(iii) Sufficient ropivacaine/adhesive premix was poured into the centre of the release liner and close to the doctor knife and the premix spread over the release liner.
(iv) The solvents were evaporated from the premix by inserting the pin frame with release liner and premix into the MATHIS laboratory coating device type LTSV to expose the solvents to a 50° C. environment for 30 min.
(v) Placebo patches were prepared according to Steps (iii) to (v) in the absence of drug.
(vi) The backing layer (ScotchPak 9730) was applied and the patch was cut into equal size portions (3×3 cm) using a patch cutter and stored at 2-8° C. for physical stability assessment.
(vii) The patches were characterised for macroscopic appearance and microscopic observations.

The microscopic observations were made using light microscopy with a 400× magnification as follows:
(i) A suitably sized patch was cut and any release liner was removed;
(ii) The patch was adhered to a glass slide and the backing layer was removed using tweezers;
(iii) The adhesive layer on the glass slide was examined using both polarised and non-polarised light for the presence of ropivacaine crystals and directly with the corresponding placebo patch; and
(iv) The microscopic observations were recorded.

The results are summarised in Table 2. The results show that ropivacaine appeared to be physically stable in all patches and for all concentrations of ropivacaine assessed, regardless of the level of silicone adhesive in the patch.

TABLE 2
Short-term physical stability of adhesive-only patches as determined
by the presence of drug crystals under polarised light

| | | | Presence of drug | |
|---|---|---|---|---|
| | | Concentration | crystals? | |
| | | of ropivacaine | T = 1 | T = 2 |
| Adhesive blend | | (% w/w) | week | week |
| Durotak | RPS1C | 8 | X | X |
| 387-2054 | 75:25 | 10 | X | X |
| | Acrylate:Silicone | 11 | X | X |
| | | 12 | X | X |
| | RPS1D | 8 | X | X |
| | 90:10 | 10 | X | X |
| | Acrylate:Silicone | 11 | X | X |
| | | 12 | X | X |
| Durotak | RPS2B | 7 | X | X |
| 87-2852 | 50:50 | 8 | X | X |
| | Acrylate:Silicone | 9 | X | X |
| | | 10 | X | X |
| | RPS2C | 10 | X | X |
| | 75:25 | 11 | X | X |
| | Acrylate:Silicone | 12 | X | X |
| | | 13 | X | X |
| | RPS2D | 10 | X | X |
| | 90:10 | 11 | X | X |
| | Acrylate:Silicone | 12 | X | X |
| | | 13 | X | X |

(✓)= drug crystals present;
(X) = drug crystals absent
Patches containing adhesive blends shown and excipient mixture (PG:Cap90:Brij93, 30:60:10)

Transdermal patches comprising varying levels of ropivacaine, the adhesive blend and the excipient mixture were prepared and those that were free of any evident drug crystals were placed onto short-term stability at 2-8° C. for t=1 and 2 weeks. The results are summarised in Table 3.

The physical stability of ropivacaine in formulations RPS2C and RPS2D was observed to be good, with no drug crystals being observed following the t=2 week time point and storage at 2-8° C. However, formulation RPS2B, which contains the highest level of silicone adhesive, showed ropivacaine instability at higher ropivacaine concentrations (9 and 10%).

TABLE 3
Short-term physical stability of adhesive-only patches as determined
by the presence of drug crystals under polarised light

| | | Concen- | | | |
|---|---|---|---|---|---|
| | | tration of | Concen- | Presence of drug | |
| | | excipient | tration of | crystals? | |
| | | mixture | ropivacaine | T = 1 | T = 2 |
| Adhesive blend | | (% w/w) | (% w/w) | week | week |
| Durotak | RPS2B | 10 | 8 | X | X |
| 87-2852 | 50:50 | | 9 | ✓ | ✓ |
| | Acrylate:Silicone | | 10 | ✓ | ✓ |
| | RPS2C | 10 | 10 | X | X |
| | 75:25 | | 12 | X | X |
| | Acrylate:Silicone | | 13 | X | X |
| | RPS2D | 15 | 10 | X | X |
| | 90:10 | | 11 | X | X |
| | Acrylate:Silicone | | 12 | X | X |
| | | | 13 | X | X |
| | | | 14 | X | Pending |
| | | | 15 | X | Pending |

(✓) = drug crystals present;
(X) = drug crystals absent

### In Vitro Release Experiments

As shown above, it was found a reduction in the amount of excipient mixture used in the transdermal formulations of the present invention led to a surprising decrease in cold flow. The inventors also discovered that changing the nature of the penetration enhancer also gave improved properties, with isopropyl palmitate being particularly preferred.

The in vitro drug release experiments were performed according to the following protocol:

### Preparation of the Receiver Fluid

The Walpole's acetate buffer pH 4.0 (1 L) was prepared as follows:
(i) Sodium acetate anhydrous (2.95 g) was weighed into a 1 L volumetric flask.
(ii) Sodium chloride (2.7 g) was transferred to the volumetric flask in

Step (i).
(iii) The volumetric flask from Step (ii) was filled to ⅔^{rd} of volume with deionised water (MilliQ, 18.2 MΩ) and left to stir at room temperature until the solids are fully dissolved.
(iv) The pH of the buffer was recorded and acetic acid (99%) was used to adjust the pH of the solution to pH 4.0.
(v) The pH 4.0 solution from Step (iv) was filled to volume and the final pH was recorded.

### In Vitro Drug Release Testing

An in vitro drug release experiment was performed using developed transdermal patch products, n=6, according to the following procedure:
(i) Individually calibrated Franz cells were employed where each cell with an average surface area and volume of approximately 2 cm² and 10 ml respectively were used.
(ii) A 2 cm² patch product was applied to 9% EVA membrane and mounted between the donor and receptor compartments of Franz cells. The receiver fluid (Walpole's acetate buffer pH 4.0) was added to the receptor compartment.
(iii) The Franz cells were equilibrated at 32° C. in a pre-calibrated water bath.
(iv) The Franz cells were occluded using Parafilm® and were protected from light.
(v) At each time point 1 mL of the receiver fluid was removed via the Franz cell side-arm using a 1 ml syringe. After each sample was removed, pre-heated receiver fluid was replaced.
(vi) The time points for this experiment were t=0, 1, 2, 4, 6, 8, 12 and 24 h.
(vii) All samples were analysed using the European Pharmacopoeia (EP) HPLC method for the quantification of ropivacaine.

Six transdermal patches were selected for in vitro drug release experiments (dried compositions are summarised in Table 4). The six patches selected are as followed:
a) RPS2B (10% excipient mixture (PP), 8% API)—this formulation is an adhesive blend of acrylate:silicone (50:50) containing Duro-tak 87-2852 and was shown to have minimal cold flow characteristics and the active patch was shown to be physically stable at 8% w/w of ropivacaine.
b) RPS2C (10% excipient mixture (PP), 13% API)—this formulation is a variant of RPS2B where the acrylate:silicone adhesive ratio was 75:25 and contains a higher drug loading of 13% API, the maximum amount incorporated where the patch was shown to physically stable at 2-8° C. for t=2 weeks.
c) RPS2A (10% excipient mixture (MPP2), 6.5% API)—this formulation is a variant of RPS2B where the acrylate:silicone ratio was 25:75 and contains a lower drug loading.
d) RPS2B (10% MPP2, 8% API)—this formulation is a variant of patch a) wherein penetration enhancer in the excipient mixture was replaced with IPP (MPP2). The levels of API will remain the same as patch a) so as to be able to directly compare the effect of the presence of MPP2 in the patch.
e) RPS1D (10% excipient mixture (PP), 8% API)—this formulation is an adhesive blend of acrylate:silicone (90:10) containing Duro-tak 387-2054 where low-med cold flow levels were observed. The API level is estimated based on previous solubility experiments on the Duro-tak 387-2054 and 10% ternary mixture.
f) RPS1G (5% excipient mixture (PP), 8% API)—this formulation is a variant of RPS1D, where the acrylate:silicone adhesive ratio was 95:5. This formulation contains a low level of the excipient mixture (PP) and a low level of silicone in an attempt to reduce cold flow.
   In a) to f) above, the API is ropivacaine.

TABLE 4
Theoretical dried compositions of final patches selected by the Sponsor for in
vitro drug release testing

| | | Theoretical dried composition (% w/w) | | | | | |
|---|---|---|---|---|---|---|---|
| | | | | | RPS2B | | |
| | | RPS2B | RPS2C | RPS2A | (10% | RPS1D | RPS1G |
| | | (10% PP; | (10% PP; | (10% PP; | MPP2; 8% | (10% PP; | (5% PP; |
| Formulation | | 8% API^{†}) | 13% API^{†}) | 6.5% API^{†}) | API^{†}) | 8% API^{†}) | 8% API^{†}) |
| Acrylate | Durotak | — | — | — | — | 73.800 | 82.650 |
| adhesive | 387-2054 | | | | | | |
| | Durotak | 41.000 | 57.750 | 20.875 | 41.000 | — | — |
| | 87-2852 | | | | | | |
| Silicone | Bio-PSA | 41.000 | 19.250 | 62.625 | 41.000 | 8.200 | 4.350 |
| adhesive | 7-4302 | | | | | | |

| | | | | | | |
|---|---|---|---|---|---|---|
| Excipient | 10.000 | 10.000 | 10.000 | — | 10.000 | 5.000 |
| mixture | | | | | | |
| (PP)* | | | | | | |
| Excipient | — | — | — | 10.000 | — | — |
| mixture | | | | | | |
| (MPP2)^{#} | | | | | | |
| Ropivacaine | 8.000 | 13.000 | 6.500 | 8.000 | 8.000 | 8.000 |

*Excipient mixture (PP) = [30/60/10] (Propylene glycol/Capryol 90/Brij93)
^{#}Excipient mixture (MPP2) = [30/60/10] (Propylene glycol/Capryol 90/isopropyl palmitate)
^{†}API = Ropivacaine

The results from the in vitro drug release experiments for the 6 selected formulations detailed above are summarised in FIGS. 1 and 2 and Table 5.

TABLE 5
Steady state release rates of ropivacaine
(API) from patches between 4-24 h.

| | Steady state release | Ranking based |
|---|---|---|
| | rate over t = | on steady |
| | 4-24 h period | state release |
| | (μg/cm²/h ± | (1 = highest, |
| Formulation | SEM, n = 6) | 6 = lowest) |
| RPS2B (10% PP; 8% API) | 1.20 ± 0.32 | 4 |
| RPS2C (10% PP; 12% API) | 2.06 ± 0.14 | 2 |
| RPS2A (10% PP; 6.5% API) | 1.35 ± 0.05 | 3 |
| RPS2B (10% MPP2; 8% API) | 2.09 ± 0.26 | 1 |
| RPS1D (10% PP; 8% API) | 0.70 ± 0.11 | 5 |
| RPS1G (5% PP; 8% API) | 0.32 ± 0.01 | 6 |
| SF1 (35% PP, 6.5% API)* | 1.94 ± 0.17 | — |
| Lidoderm* | 3.24 ±0.57 | — |

While specific embodiments of the invention have been described herein for the purpose of reference and illustration, various modifications will be apparent to a person skilled in the art without departing from the scope of the invention as defined by the appended claims.

What is claimed is:

1. A transdermal patch comprising a pharmaceutical formulation, said formulation comprising:
(i) ropivacaine or an opioid,
(ii) a pharmaceutically-acceptable adhesive comprising up to 75% (w/w) silicone or polyisobutylene, and
(iii) between 5 and 20% w/w of an excipient mixture comprising, a hydrophilic material, a penetration enhancer and a carrier oil having a ropivacaine or an opioid solubility of greater than or equal to 1.5% (w/w).

2. The transdermal patch of claim 1, wherein the pharmaceutical formulation has an in vitro human skin permeation rate of the ropivacaine or opioid that is greater than 1.8 μg cm⁻² h⁻¹.

3.-5. (canceled)

6. The transdermal patch of claim 1, wherein the amount of the ropivacaine or an opioid is between 3 and 20% w/w.

7.-8. (canceled)

9. The transdermal patch of claim 1, wherein the adhesive has a ropivacaine or an opioid solubility greater than 2.5% w/w at room temperature.

10. The transdermal patch of claim 1, wherein the amount of adhesive is 60 to 92% w/w.

11. The transdermal patch of claim 1, wherein the adhesive is an acrylate material or a blend of acrylate materials or rubbers comprising up to 75% (w/w of the adhesive) silicone or polyisobutylene.

12. The transdermal patch of claim 1, wherein the adhesive is an acrylate material or a blend of acrylate materials and silicone, wherein the ratio of acrylate:silicon is between 95:5 to 25:75.

13.-18. (canceled)

19. The transdermal patch of claim 1, wherein the ratio of hydrophilic material:carrier oil:penetration enhancer in the excipient mixture is 20-40:40-60:5-20.

20. (canceled)

21. The transdermal patch of claim 1, wherein the carrier oil is present at an amount of 1 to 15% w/w of the pharmaceutical formulation.

22.-23. (canceled)

24. The transdermal patch of claim 21, wherein the carrier oil has a water solubility of less than 0.1% w/w and a ropivacaine or opioid solubility in excess of 3% w/w.

25. (canceled)

26. The transdermal patch of claim 21, wherein the carrier oil is selected from the group consisting of sorbitan monooleate, sorbitan trioleate, triglycerides of caprylic/capric acid, propylene glycol dicaprylate/dicaprate, ethoxy diglycol, propylene glycol monocaprylate, glycerol monooleate, lanolin, acetylated lanolin, polyethylene glycol lanolin, glycerol monocaprylate/caprate, propylene glycol laurate, and/or mono- or diglycerides of capric acid.

27.-28. (canceled)

29. The transdermal patch of claim 1, wherein the penetration enhancer is present in an amount of 0.5 to 15% w/w of the pharmaceutical formulation.

30. (canceled)

31. The transdermal patch of claim 29, wherein the penetration enhancer is polyoxyethylene oleyl ether or a compound of the formula I, shown below: 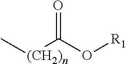
(I)
wherein;
R₁ is a (1-6C)alkyl; and
n is an integer of between 6 and 20.

32. (canceled)

33. The transdermal patch of claim 1, wherein the hydrophilic material is present at an amount of 1 and 15% w/w of the pharmaceutical formulation.

34. (canceled)

35. The transdermal patch of claim 33, wherein the hydrophilic material is selected from the group consisting of propylene glycol, glycerol, polyethylene glycol, short chain water soluble esters of citric acid, acetic acid, hexylene glycol and alcohols, including diols and polyols.

36. (canceled)

37. The transdermal patch of claim 1, wherein the excipient mixture further comprises an additive selected from the group consisting of non-ionic surfactants, hydrophilic surfactants, terpenes and membrane disruptors.

38. The transdermal patch of claim 37, wherein the additive is selected from the group consisting of menthol and diethylene glycol monoethyl ether.

39. The transdermal patch according to of claim 1, wherein the patch comprises ropivacaine or oxycodone.

40.-44. (canceled)

45. A method of treating pain, said method comprising topically applying the transdermal patch of claim 1 to a human or animal subject in need of such treatment.

46. The method as claimed in claim 45, wherein the type of pain is neuropathic and/or nociceptive pain.

47. (canceled)
